(19) United States
(12) Patent Application Publication
(10) Pub. No.: US 2005/0203601 A1
(43) Pub. Date: Sep. 15, 2005

(54) Neural stimulation array providing proximity of electrodes to cells via cellular migration

(76) Inventors: Daniel Palanker, Sunnyvale, CA (US); Philip Huie, Cupertino, CA (US); Alexander Vankov, Mountain View, CA (US); Harvey Fishman, Menlo Park, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/035,692

(22) Filed: Jan. 13, 2005

Related U.S. Application Data
(63) Continuation-in-part of application No. 10/742,584, filed on Dec. 19, 2003.
(60) Provisional application No. 60/447,796, filed on Feb. 14, 2003.
(60) Provisional application No. 60/447,421, filed on Feb. 14, 2003.
(60) Provisional application No. 60/538,947, filed on Jan. 22, 2004.

(51) Int. Cl. A61N 1/00; A61N 1/04; A61N 1/05

(52) U.S. Cl. 607/116

(57) ABSTRACT

An interface for selective excitation or sensing of neural cells in a biological neural network is provided. The interface includes a membrane with a number of channels passing through the membrane. Each channel has at least one electrode within it. Neural cells in the biological neural network grow or migrate into the channels, thereby coming into close proximity to the electrodes. Once one or more neural cells have grown or migrated into a channel, a voltage applied to the electrode within the channel selectively excites the neural cell(s) in that channel. The excitation of these neural cell(s) will then transmit throughout the neural network (i.e. cells and axons) that is associated with the neural cell(s) stimulated in the channel. An alternative interface provides cell excitation via an array of electrically conductive pillars on a substrate. The pillars have electrically insulated sides and exposed top surfaces, to provide selective cell excitation.

### CROSS REFERENCE TO RELATED APPLICATIONS

This application is a continuation in part of U.S. patent application Ser. No. 10/742,584, filed Dec. 19, 2003 and entitled “Interface for Making Spatially Resolved Electrical Contact to Neural Cells in a Biological Neural Network”, which claims the benefit of U.S. provisional applications 60/447,796 and 60/447,421, both filed on Feb. 14, 2003.

This application claims the benefit of U.S. provisional application 60/538,947, filed on Jan. 22, 2004, entitled “Neural Stimulation Array Providing Proximity of Electrodes to Cells via Cellular Migration”, and hereby incorporated by reference in its entirety.

### FIELD OF THE INVENTION

The present invention relates generally to electrical stimulation or sensing of neural cells. More particularly, the present invention relates to an electrode configuration for selectively making electrical contact to neural cells.

### BACKGROUND

Several degenerative retinal diseases that commonly lead to blindness, such as retinitis pigmentosa and age-related macular degeneration, are primarily caused by degradation of photoreceptors (i.e., rods and cones) within the retina, while other parts of the retina, such as bipolar cells and ganglion cells, remain largely functional.

Accordingly, an approach for treating blindness caused by such conditions that has been under investigation for some time is provision of a retinal prosthesis connected to functional parts of the retina and providing photoreceptor functionality.

Connection of a retinal prosthesis to functional parts of the retinal is typically accomplished with an array of electrodes (see, e.g., U.S. Pat. No. 4,628,933 to Michelson). Michelson teaches a regular array of bare electrodes in a “bed of nails” configuration, and also teaches a regular array of coaxial electrodes to reduce crosstalk between electrodes. Although the electrodes of Michelson can be positioned in close proximity to retinal cells to be stimulated, the electrode configurations of Michelson are not minimally invasive, and damage to functional parts of the retina may be difficult to avoid.

Alternatively, a prosthesis having electrodes can be positioned epiretinally (i.e., between the retina and the vitreous humor) without penetrating the retinal internal limiting membrane (see, e.g., U.S. Pat. No. 5,109,844 to de Juan et al.). Although the arrangement of de Juan et al. is less invasive than the approach of Michelson, the separation between the electrodes of de Juan et al. and retinal cells to be stimulated is larger than in the approach of Michelson.

Such increased separation between electrodes and cells is undesirable, since electrode crosstalk and power required to stimulate cells both increase as the separation between electrodes and cells increases. Furthermore, increased electrical power has further undesirable effects such as increased resistive heating in biological tissue and increased electrochemical activity at the electrodes.

U.S. Pat. No. 3,955,560 to Stein et al. is an example of an approach which provides low separation between electrodes and nerve fibers (i.e., axons), but requires a highly invasive procedure where a nerve is cut and then axons regenerate through a prosthesis and past electrodes embedded within the prosthesis.

Another approach for making electrical contact to cells is considered in U.S. Pat. No. 6,551,849 to Kenney. In this approach, an array of needles is formed on a silicon substrate by lithographic techniques. However, as in the Michelson reference above, insertion of such an array of needles into tissue is not minimally invasive. Furthermore, the sides of the silicon needles of Kenney are exposed and can make electrical contact to cells, which undesirably reduces the spatial precision of cellular excitation.

### OBJECTS AND ADVANTAGES

Accordingly, an objective of the present invention is to provide apparatus and method for selectively making electrical contact to neural cells with electrodes in close proximity to the cells and in a minimally invasive manner.

Another objective of the present invention is to instigate or allow migration of the neural cells towards the stimulating electrodes in order to minimize the distance between an electrode and a cell.

Yet another objective of the present invention is to preserve functionality of a biological neural network when instigating or allowing migration of neural cells.

Still another objective of the present invention is to reduce cross-talk between neighboring electrodes.

Another objective of the present invention is to ensure low threshold voltage and current for cell excitation.

Yet another objective of the present invention is to provide an interface that allows for mechanical anchoring of neural tissue to a prosthesis.

Still another objective of the present invention is to provide a large electrode surface area to decrease current density and thereby decrease the rate of electrochemical erosion.

An advantage of the present invention is that a selected cell or group of neural cells can be brought into proximity to stimulating or sensing electrodes while preserving the signal processing functionality of a biological neural network. A further advantage of the present invention is that by bringing cells into close proximity to electrodes, electrical power required for cell excitation is reduced, thus decreasing tissue heating and electrode erosion. Another advantage of the present invention is that close proximity between cells and electrodes reduces cross-talk with non-selected cells, thus allowing a higher packing density of electrodes which provides improved spatial resolution.

### SUMMARY

The present invention provides an interface for selective excitation or sensing of neural cells in a biological neural network. The interface includes a membrane with a number of channels passing through the membrane. Each channel has at least one electrode within it. Neural cells in the biological neural network grow or migrate into the channels, thereby coming into close proximity to the electrodes.

Once one or more neural cells have grown or migrated into a channel, a voltage applied to the electrode within the channel selectively excites the neural cell (or cells) in that channel. The excitation of these neural cell(s) will then transmit throughout the neural network (i.e., cells and axons) that is associated with the neural cell(s) stimulated in the channel. Alternatively, excitation of a neural cell (or cells) within the channel due to activity within the biological neural network is selectively sensed by the electrode within the channel.

An alternative embodiment of the invention provides cell excitation via an array of electrically conductive pillars on a substrate. The pillars have electrically insulated sides and exposed top surfaces, to provide selective cell excitation. More specifically, cells separated from the top surface of the pillar by a distance comparable to (or less) than the radius of the pillar are excited. Pillars are separated by distances sufficient for cellular migration in between them, thus providing slow and non-disruptive penetration to a predetermined depth into tissue.

### BRIEF DESCRIPTION OF THE DRAWINGS

FIG. 1 shows an embodiment of the invention having a membrane with channels positioned under a retina.

FIG. 2 shows an embodiment of the invention having a membrane with channels positioned under a retina, and having cells from the inner nuclear layer migrated into the channels.

FIG. 3 shows a side view of an embodiment of the invention having a membrane with an electrode exposed inside the channel and coated outside the channel at the bottom of the membrane.

FIG. 4 shows a bottom view of an embodiment of the invention according to FIG. 3.

FIG. 5 shows an embodiment of the invention having a membrane with channels positioned under a retina, and having neural cells migrated into the channels. Voltage applied to a channel electrode causes excitation of neural cells in that channel. The excited neural cells in that channel transmit signal(s) to the retinal network.

FIG. 6 shows an embodiment of the invention having channels with two different channel diameters, and having a stop layer at the bottom to prevent cell migration past the channel while allowing nutrient flow.

FIG. 7 shows an embodiment of an array according to the present invention.

FIG. 8 shows an embodiment of the invention where only a few (ideally one) neural cells can enter the channel. An electric field is applied across the cell providing efficient stimulation.

FIG. 9 shows an embodiment of the invention having an electrode and/or an insulator laterally extending into a channel.

FIG. 10 shows an embodiment of the invention having photosensitive circuitry connected to the electrodes, and having a perforated stop layer at the bottom to prevent cell migration past the channel while allowing nutrient flow.

FIG. 11 shows an embodiment of the invention having electrodes disposed on channel end faces.

FIGS. 12a-b show embodiments of the invention having pillars for making selective electrical contact to cells.

### DETAILED DESCRIPTION OF THE DRAWINGS

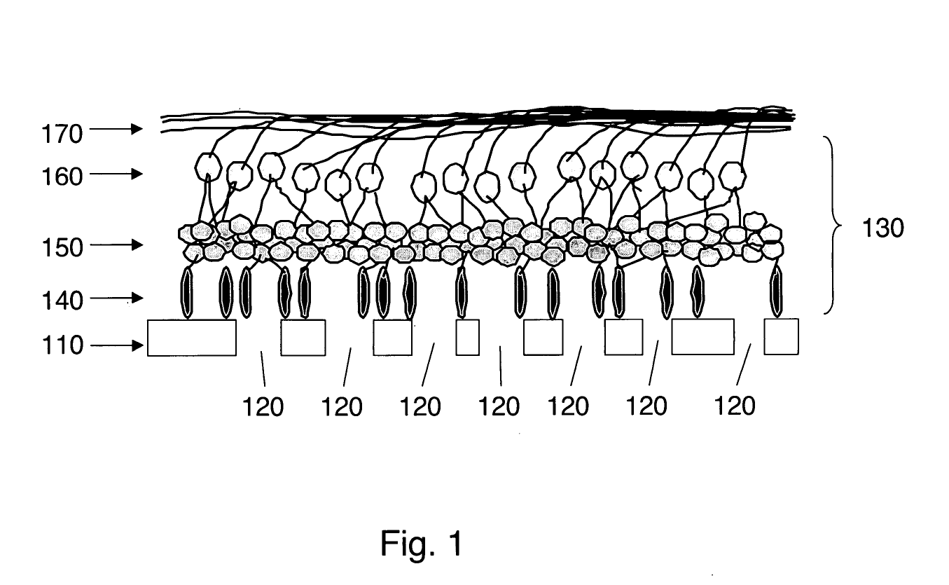

FIG. 1 shows an embodiment of the invention having a membrane 110 with a plurality of channels 120 passing through membrane 110. In the example of FIG. 1, membrane 110 is preferably positioned under a retina 130. Exemplary retina 130 includes photoreceptors (i.e., rods and/or cones) 140, inner nuclear layer cells 150 (e.g., bipolar cells), ganglion cells 160 and respective axons connecting to an optic nerve 170. Membrane 110 can be of any type of biocompatible material that is substantially electrically non-conductive and is flexible enough to conform to the shape of the neural tissue in a biological neural network. Suitable materials for membrane 110 include mylar and PDMS (polydimethylsiloxane). The thickness of membrane 110 is less than 0.5 mm, and is preferably between about 5 microns and about 100 microns. Channels 120 pass completely through membrane 110 and can be of any shape, although substantially circular shapes are preferred. Retina 130 on FIG. 1 is an example of a biological neural network. The invention is applicable to making electrical contact to any kind of biological neural network, including but not limited to: central nervous system (CNS) neural networks (e.g., brain cortex), nuclei within the CNS, and nerve ganglia outside the CNS. A biological neural network is made up of interconnected biological processing elements (i.e., neurons) which respond in parallel to a set of input signals given to each.

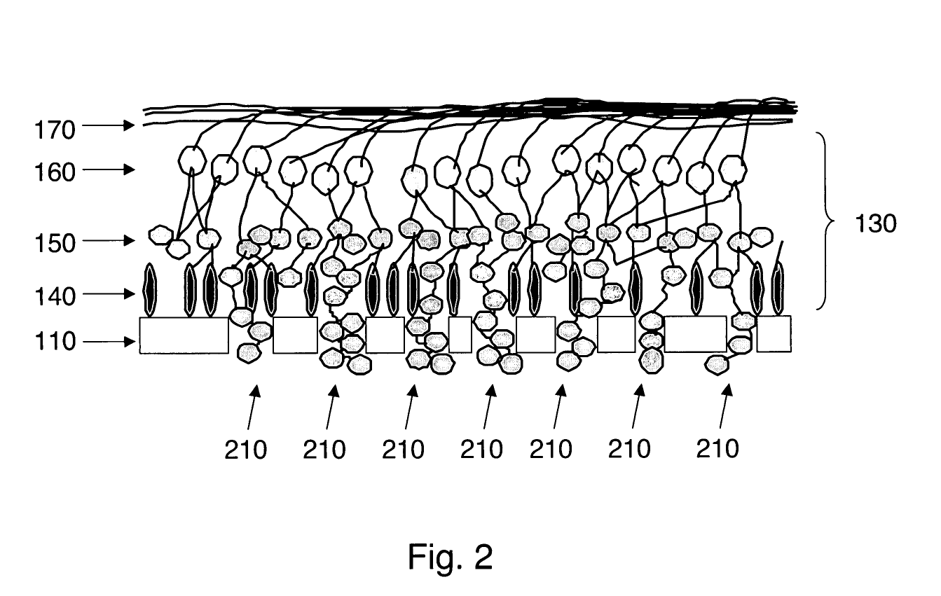

FIG. 2 shows cell migration into channels 120 of membrane 110 of FIG. 1. When membrane 110 is positioned near a layer of neural tissue, neural cells in the neural tissue layer will tend to grow or migrate towards the channels. This growth process is a natural physiological response of cells and may depend on the existence of nutrients, space and a suitable surface morphology for these cells. Optionally, a growth (or inhibition) factor could be included to enhance (or decrease) the migration or growth of the neural cells. Such factors include but are not limited to: BDNF (brain-derived neurotrophic factor, CNTF (ciliary neurotrophic factor), Forskolin, Laminin, N-CAM and modified N-CAMs. However, such a growth or inhibition factor is not always necessary. In the example of FIG. 2, cells 210 are neural cells 150 which have migrated into and/or through channels 120 in membrane 110 positioned subretinally. The diameter of each channel should be sufficient to allow migration of neural cells 150, and is preferably in a range from about 5 microns to about 20 microns. We have found experimentally that such cell migration tends to occur easily when membrane 110 is disposed subretinally (i.e. between the retina and the outer layers of the eye), and tends not to occur easily (or at all) when membrane 110 is disposed epiretinally (i.e. between the retina and the vitreous humor). Penetration of neural cells 150 into and through channels 120 provides mechanical anchoring of retina 130 to membrane 110.

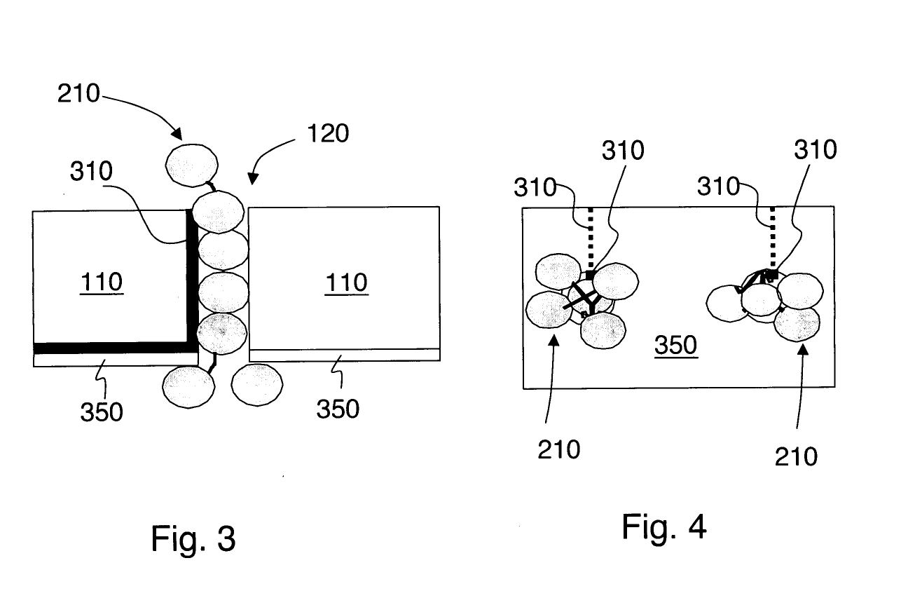

FIG. 3 shows an enlarged view of one of the channels of the configuration of FIG. 2. In the example of FIG. 3, an electrode 310 is positioned inside channel 120 in membrane 110 leaving enough space for neural cells 210 and their axons to migrate and grow through the channel. As a result of this cell migration, electrode 310 is in close proximity to neural cells 210. Electrode 310 is shown extending to a bottom surface of membrane 110 (i.e., a surface of membrane 110 facing away from the biological neural network). Wires (not shown) can connect electrodes 310 to input and/or output terminals (not shown), or to circuitry within membrane 110. In such cases where electrodes 310 and optionally wires are present on the bottom surface of membrane 110, a non-conductive layer 350 is preferably disposed on the bottom surface of membrane 110 covering electrodes 310 (and any wires, if present) to provide electrical isolation. FIG. 4 shows a view as seen looking up at non-conductive layer 350 of two channels 120 having the configuration of FIG. 3. FIG. 4 also shows close proximity between electrodes 310 and cells 210.

Electrodes 310 are in electrical contact with neural cells 210, but may or may not be in physical contact with neural cells 210. Direct physical contact between electrodes 310 and cells 210 is not necessary for electrodes 310 to stimulate cells 210, or for electrodes 310 to sense activity of cells 210.

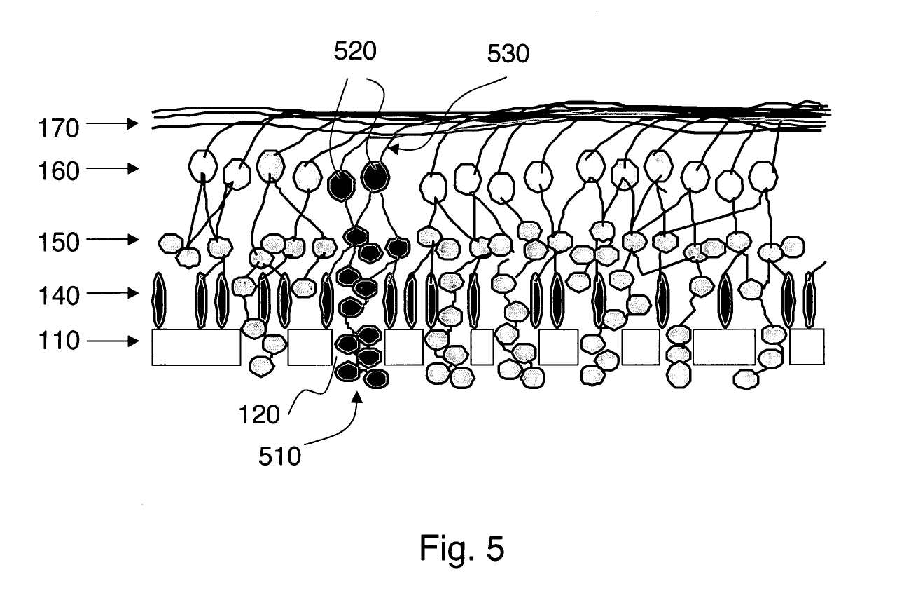

FIG. 5 shows operation of the configuration of FIG. 2. A selected neural cell (or cells) 510 within one of channels 120 is electrically excited by an electrode within the same channel. Impulses from neural cell (or cells) 510 excite selected ganglion cells 520, which in turn excite selected optic nerve fibers 530.

Many advantages of the present invention are provided by the configurations discussed in connection with FIGS. 1-5. In particular, close proximity between electrodes 310 and migrated cells 210 is provided, which reduces the electrical power required to stimulate cells 210 and decreases cross-talk to unselected cells (i.e., cells not within the channel 120 corresponding to a particular electrode 310). Reduction of electrical power required to stimulate cells 210 leads to reduced tissue heating and to reduced electrochemical erosion of electrodes 310. Reduction of cross-talk to unselected cells provides improved spatial resolution. Furthermore, electrodes 310 are well insulated from each other by membrane 110, so electrode to electrode cross-talk is also reduced. Additionally, the growth and/or migration of neural cells 150 into channels 120 preserves existing functionality of retina 130.

However, the configurations shown in FIGS. 1-5 do not directly limit growth and/or migration of cells through channels 120. In some cases, we have found that many cells grow or migrate through channels 120, leading to the formation of significant uncontrolled “tufts” of cells and/or cell processes facing away from the retina. Such uncontrolled tuft growth can lead to fusing of adjacent tufts, which tends to undesirably increase crosstalk. Also, electrodes 310 have a small surface area, which increases current density and thus increases undesirable electrochemical activity at electrodes 310.

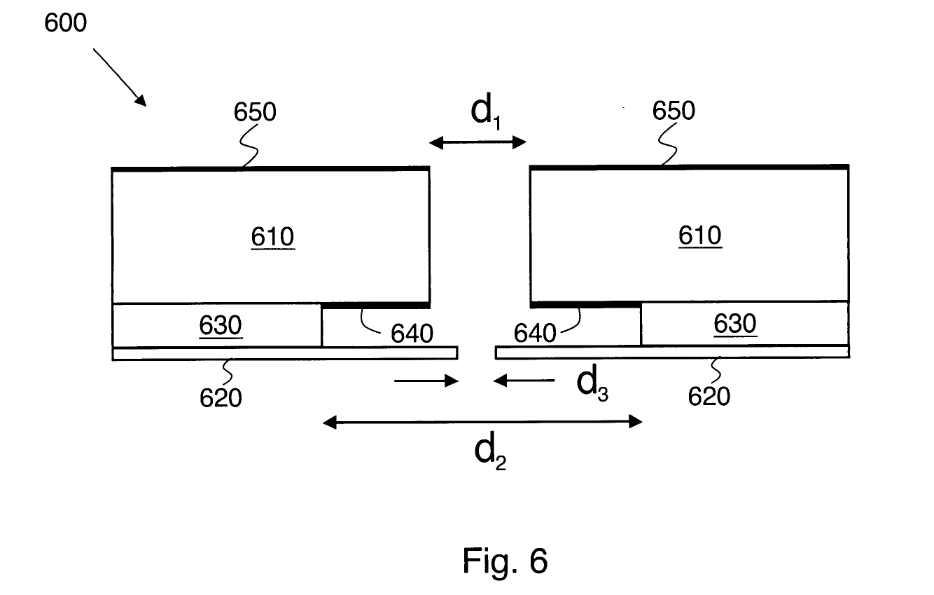

FIG. 6 shows an interface 600 according to an embodiment of the invention which prevents the formation of such uncontrolled retinal tufts and provides increased electrode surface area. In the embodiment of FIG. 6, a first layer 610 and a second layer 630 form a membrane analogous to membrane 110 of FIG. 1. A channel passes through both first layer 610 and second layer 630, where the channel diameter d2 in second layer 630 is larger than the channel diameter d1 in first layer 610. The thickness of layers 610 and 630 together is less than 0.5 mm. The thickness of layer 610 is preferably between about 10 microns and about 50 microns. The thickness of layer 630 is preferably between about 5 microns and about 50 microns. A stop layer 620 is disposed such that second layer 630 is in between first layer 610 and stop layer 620. Stop layer 620 is shown as having a hole with diameter d3 aligned to the channel through layers 610 and 630. An electrode 640 is disposed on a surface of first layer 610 facing second layer 630.

Layers 610, 620, and 630 can be of any type of biocompatible material that is substantially electrically non-conductive and is flexible enough to conform to the shape of the neural tissue in a biological neural network. Suitable materials include mylar and PDMS (polydimethylsiloxane).

First layer 610 is in proximity to and faces a biological neural network (not shown on FIG. 6). Retina 130 as shown on FIG. 1 is an example of such a biological neural network. As discussed above in connection with FIG. 2, cells tend to grow or migrate into channels within layer 610, provided there is sufficient room. Accordingly, the diameter d1 should be sufficiently large to allow migration of neural cells (such as 150 on FIG. 1), and is preferably in a range from about 5 microns to about 50 microns.

The function of stop layer 620 is to prevent uncontrolled growth of a retinal tuft past stop layer 620, while permitting nutrients to flow to a cell (or cells) within the channel passing through layers 610 and 630. Therefore, diameter d3 should be small enough to prevent growth or migration of cells (or cell process) through stop layer 620. Preferably, d3 is less than about 5 microns in order to prevent cell migration through stop layer 620. Alternatively, stop layer 620 can include several small holes each having a diameter of less than about 5 microns, where the holes in layer 620 are aligned with the channel within second layer 630. More generally, stop layer 620 can be either an impermeable membrane having at least one hole in it large enough to permit nutrient flow and small enough to prevent cells from moving through it, or a membrane which is permeable to nutrient flow.

Since diameter d2 is larger than diameter d1, a retinal tuft may form within the channel through second layer 630. Such retinal tuft formation is not uncontrolled, since the maximum size of the retinal tuft is determined by stop layer 620. In fact, controlled retinal tuft formation is likely to be desirable, since it will tend to provide improved mechanical anchoring of interface 600 to a retina.

Electrode 640 is disposed on a surface of first layer 610 facing second layer 630 and within the channel passing through the two layers. Since d2 is greater than d1, the surface area of electrode 640 can be made significantly larger than the area of an electrode within a channel having a uniform channel diameter along its length (such as shown on FIG. 3). The diameter d2 is preferably from about 10 microns to about 100 microns. In the example of FIG. 6, an electrode 650 is disposed on the top surface of first layer 610. An applied voltage between electrodes 640 and 650 provides an electric field within the channel passing through first layer 610.

One variation of the present invention is to coat electrode 640 to further increase its surface area and to further decrease the current density and associated rate of electrochemical erosion of the conductive layer. For example, carbon black has a surface area of about 1000 m²/g and so a coating of carbon black on electrode 640 can significantly increase its effective surface area. Other suitable materials for such a coating include platinum black, iridium oxide, and silver chloride.

Laser processing can be used to form channels. In the case of the embodiment of FIG. 6, the largest holes (i.e. the channels through second layer 630) are formed first, then layers 630 and 610 are attached to each other. The next largest holes are then formed, using the previously formed holes for alignment, and stop layer 620 is then attached to second layer 630. Finally, the smallest holes (if necessary) are formed in stop layer 620, using previously formed holes for alignment. Electrodes 640 on first layer 610 can also be formed by laser processing. For example, first layer 610 can have a continuous film of metal deposited on the surface of layer 610 that will eventually face toward second layer 630, and laser processing of this continuous film of metal can define electrodes 640 (and optionally wires connected to these electrodes as discussed in connection with FIG. 3). Laser processing methods to perform these tasks are known in the art.

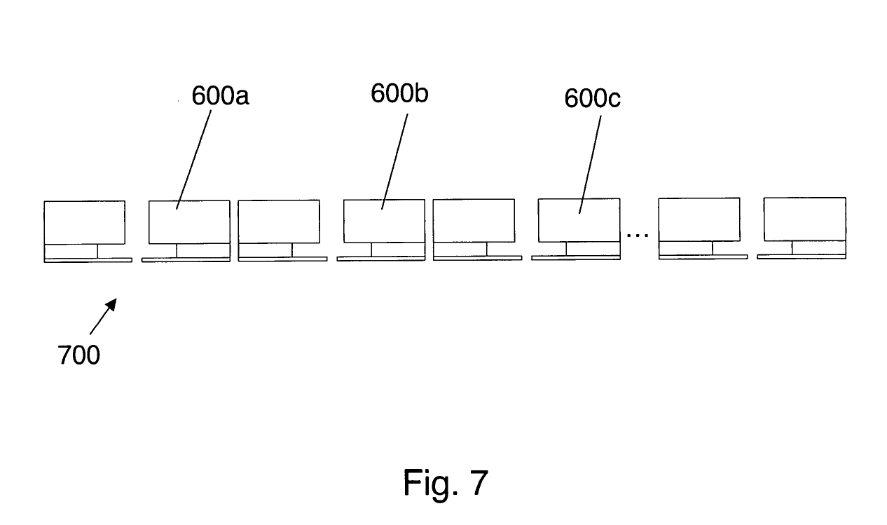

FIG. 7 shows an interface 700 including several interfaces 600 (shown as 600a, 600b, 600c, etc.) according to FIG. 6, for making selective contact to multiple points in a retina. Typically, interfaces 600 within interface 700 are arranged as a two-dimensional array, where each channel corresponds to a pixel of the array. In the embodiment of FIG. 7, electrode 650 is preferably a common electrode for all channels. Resistance between electrodes 640 corresponding to different array elements is largely determined by the diameter d3 of the hole in stop layer 620, since conduction is mainly through extra cellular fluid surrounding interfaces 600. Accordingly, the selection of d3 (or equivalently, the total open area in stop layer 620) is determined by a tradeoff between reducing electrode to electrode cross-talk (by decreasing d3) and providing sufficient nutrient flow (by increasing d3).

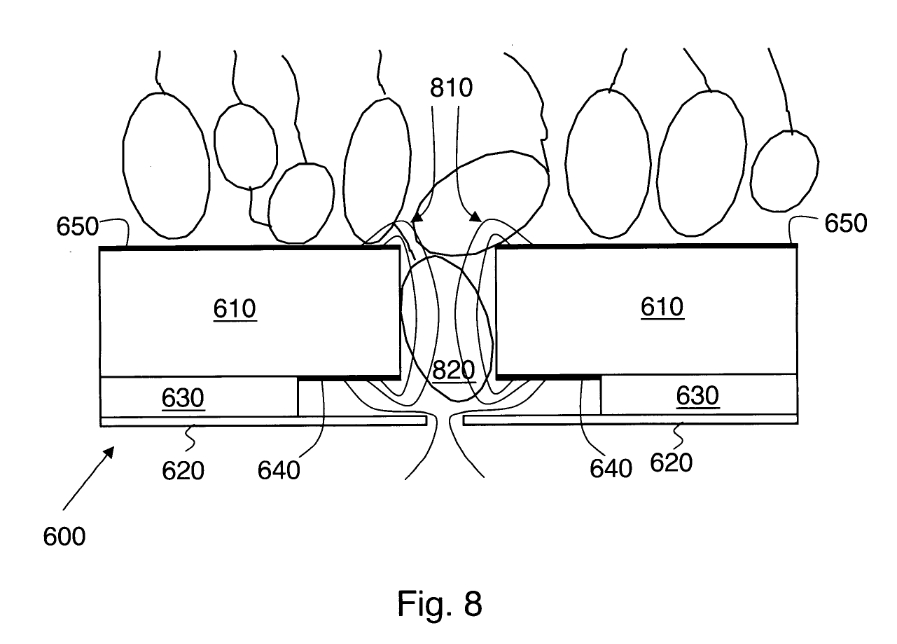

FIG. 8 shows operation of interface 600, where a single cell 820 has migrated into the channel passing through first layer 610. In practice, several cells may be present in this channel, although the ideal situation of having only a single cell in the channel is preferred because it provides maximum selectivity of excitation. A potential difference between electrodes 640 and 650 creates an electric field 810 passing through cell 820 as shown. Electric field 810 depolarizes cell 820 to stimulate it, and the resulting signal travels into the rest of the retina as indicated in FIG. 5.

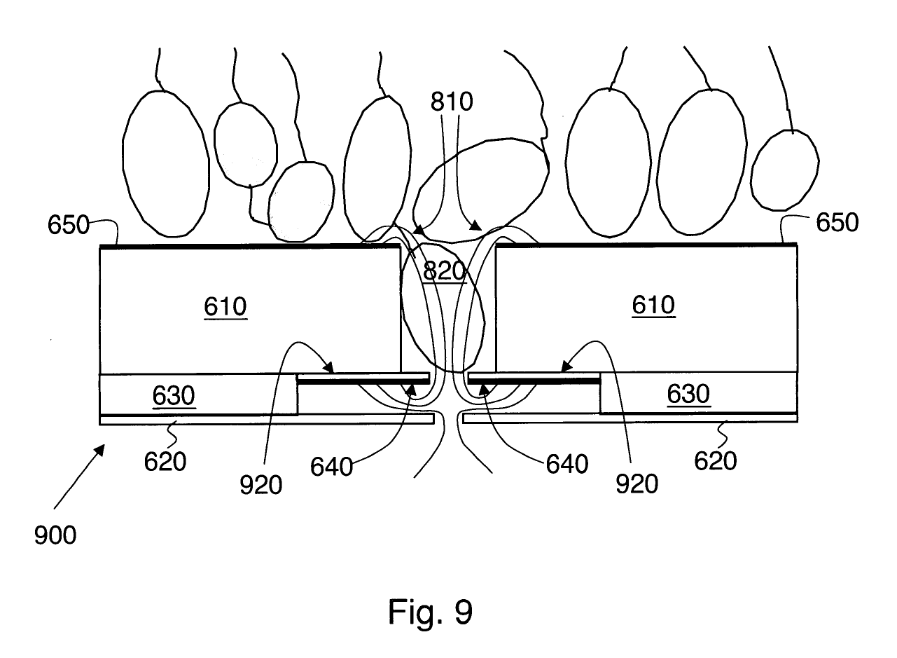

FIG. 9 shows operation of an interface 900 which is a variation of interface 600. In interface 900, electrode 640 and/or an insulating intermediate layer 920 is/are extended partway into the channel passing through first layer 610. The example of FIG. 9 shows both electrode 640 and intermediate layer 920 extending into the channel. Such reduction of the minimum channel diameter reduces the electrical power required to excite cell 820, because the impedance of electrode 640 increases. A part of the cell 820 located close to the small opening in electrode 640 and intermediate layer 920 will be depolarized. Extension of electrode 640 in this manner also further increases its surface area, which desirably reduces the rate of electrochemical erosion of electrode 640.

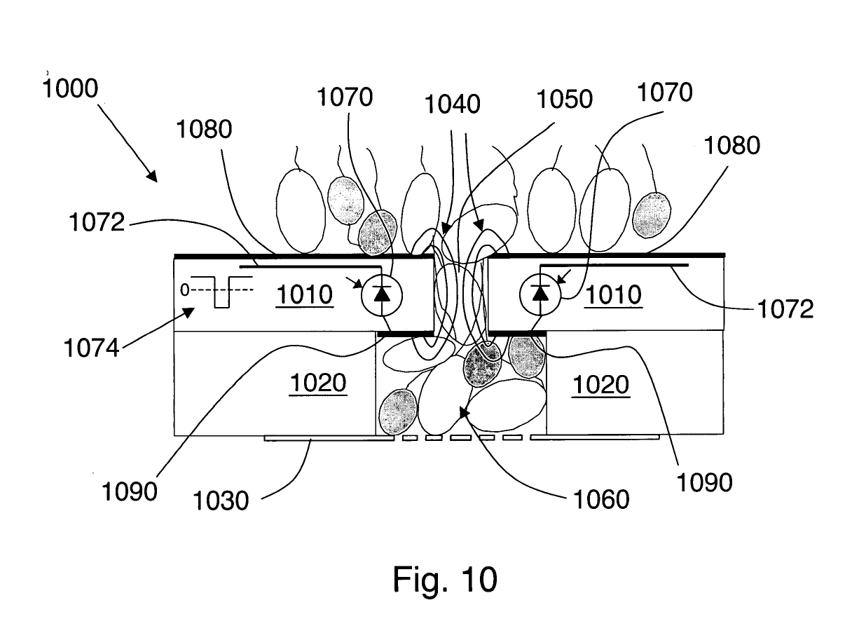

FIG. 10 shows operation of an interface 1000 according to another embodiment of the invention. In the embodiment of FIG. 10, a first layer 1010 and a second layer 1020 form a membrane analogous to membrane 110 of FIG. 1. A channel passes through both first layer 1010 and second layer 1020, where the channel diameter in second layer 1020 is larger than the channel diameter in first layer 1010. The thickness of layers 1010 and 1020 together is less than 0.5 mm. As shown on FIG. 10, the thickness of second layer 1020 is on the order of several times a typical cell dimension, to provide room for formation of a controlled retinal tuft within second layer 1020. Layer 1010 preferably has a thickness between about 5 microns and about 50 microns. Layer 1020 preferably has a thickness between about 5 microns and about 100 microns. A stop layer 1030 is disposed such that second layer 1020 is in between first layer 1010 and stop layer 1030.

The function of stop layer 1030 is to prevent uncontrolled growth of a retinal tuft past stop layer 1030, while permitting nutrients to flow to a cell (or cells) within the channel passing through layers 1010 and 1020. Stop layer 1030 is shown as having several small holes aligned to the channel through layer 1020. Preferably, these holes each have a diameter of less than about 5 microns, to prevent cell migration through the holes. Alternatively, stop layer 1030 could have a single small hole per channel, as shown on FIG. 6. More generally, stop layer 1030 can be either an impermeable membrane having at least one hole in it large enough to permit nutrient flow and small enough to prevent cells from moving through it, or a membrane which is permeable to nutrient flow.

An electrode 1090 is disposed on a surface of first layer 1010 facing second layer 1020, and another electrode 1080 is disposed on a surface of first layer 1010 facing away from second layer 1020. A photo-sensitive circuit 1070 (e.g., a photodiode, a phototransistor, etc.) is fabricated within first layer 1010 and is connected to electrodes 1080 and 1090. Electrode 1080 is preferably transparent to light and/or patterned in such a way that allows for light penetration to photo-sensitive circuit 1070. Electrode 1080 is also preferably common to all channels.

The embodiment of FIG. 10 provides photo-sensitive circuit 1070 connected to electrodes 1090. Accordingly, it is preferable for layer 1010 to be fabricated from a light-sensitive material permitting fabrication of photo-sensitive circuitry 1070 (e.g., any of various compound semiconductors such as GaAs and the like). Furthermore, for this embodiment, it is convenient for layers 1020 and 1030 to be materials compatible with the processing technology of the material of layer 1010. For example, layers 1020 and 1030 can be polymers (e.g., photoresists) or inorganic materials (e.g., oxides or nitrides). Channels through layers 1010 and 1020 (and holes through layer 1030) are preferably formed via lithography, in order to enable rapid fabrication of devices having a large number of channels. Since the materials indicated above are not typically bio-compatible, biological passivation of embodiments of the invention made with such materials is preferred. Suitable biological passivation techniques for such materials are known in the art.

In operation of interface 1000, light impinging on photo-sensitive circuit 1070 leads to generation of a potential difference between electrodes 1080 and 1090. Optionally, electronic amplification of the signal of photo-sensitive circuit 1070 is provided by amplification circuitry (not shown) to increase the signal at electrodes 1080 and 1090 responsive to illumination of photo-sensitive circuit 1070. The potential difference between electrodes 1080 and 1090 provides an electric field 1040 passing through a cell 1050 within the channel. Excitation of cell 1050 by electric field 1040 provides selective excitation of the retina, as shown on FIG. 5.

Electrical excitation of electrodes 1090 is preferably delivered as bi-phasic electrical pulses. For example, a power line 1072 carrying bi-phasic pulses 1074 can deliver bi-phasic electrical current pulses to stimulating electrode 1090 subject to control by photo-sensitive element 1070. A current flows (approximately along electric field lines 1040) between stimulating electrode 1090 and return electrode 1080.

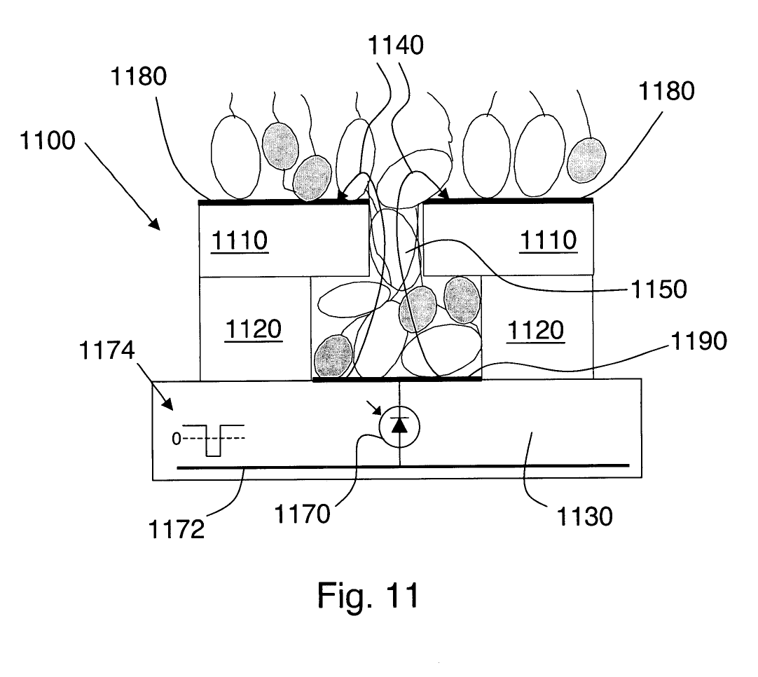

FIG. 11 shows an alternative embodiment of the invention that is similar to the embodiment of FIG. 10 except for the positioning of the channel electrodes. In interface 1100 of FIG. 11, a first layer 1110 and a second layer 1120 form a membrane analogous to membrane 110 of FIG. 1. A channel passes through both first layer 1110 and second layer 1120, where the channel diameter in second layer 1120 is larger than the channel diameter in first layer 1110. The thickness of layers 1110 and 1120 together is less than 0.5 mm. As shown on FIG. 11, the thickness of second layer 1120 is on the order of several times a typical cell dimension, to provide room for formation of a controlled retinal tuft within second layer 1120. Layer 1110 preferably has a thickness between about 5 microns and about 50 microns. Layer 1120 preferably has a thickness between about 5 microns and about 100 microns. A substrate 1130 is disposed beneath and in contact with second layer 1120.

An electrode 1190 is disposed on a surface of substrate 1130 facing the channel through first layer 1110 and second layer 1120. Thus substrate 1130 provides an end face for the channels, and electrode 1190 is disposed on this end face. In this embodiment, numerous channels are typically fabricated, each channel having an end face formed by substrate 1130 and a corresponding electrode on the end face. Another electrode 1180 is disposed on a surface of first layer 1110 facing away from second layer 1120. A photo-sensitive circuit 1170 (e.g., a photodiode, a phototransistor, etc.) is fabricated within substrate 1130 and is connected to electrode 1190. Electrode 1180 is preferably transparent to light and/or patterned in such a way that allows for light penetration to photo-sensitive circuit 1170. Electrode 1180 is also preferably common to all channels. Operation of the photo-sensitive embodiment of FIG. 11 is similar to operation of the embodiment of FIG. 10. Interface 1100 provides selective excitation of cells (e.g., cell 1150) in the narrow part of the channel (i.e., through first layer 1110) because current flow (e.g., a current 1140) between electrodes 1180 and 1190 is more concentrated in the narrow part of the channels than in the wide part of the channels.

Electrical excitation of electrodes 1190 is preferably delivered as bi-phasic electrical pulses. For example, a power line 1172 carrying bi-phasic pulses 1174 can deliver bi-phasic electrical current pulses to stimulating electrode 1190 subject to control by photo-sensitive element 1170. Current 1140 flows between stimulating electrode 1190 and return electrode 1180.

The embodiment of FIG. 11 advantageously reduces fabrication complexity, since no individually addressable circuitry is required within the membrane formed by first layer 1110 and second layer 1120. Instead, the individually addressable circuitry (i.e., electrodes 1190 and optionally photo-sensitive circuits 1170) is included in substrate 1130, which can be efficiently fabricated with standard electronic circuit manufacturing processes (since substrate 1130 has no perforations). Since the membrane formed by layers 1110 and 1120 includes only electrode 1180 (which is common to all pixels), fabrication of this membrane is significantly simplified. The membrane and substrate 1130 can be fabricated separately and integrated in a final assembly step. Alternatively, the membrane can be fabricated lithographically on top of substrate 1130 after the circuitry and electrodes of substrate 1130 have been conventionally defined.

In some cases, cells blocked in the pores of the embodiment of FIG. 11 may change their phenotype (or even die) over time. Another undesirable possibility is that electrically inactive cells may preferentially migrate into these pores (e.g., the glial or Mueller cells may migrate more rapidly than neural cells, thereby filling up the pores with relatively inactive cells).

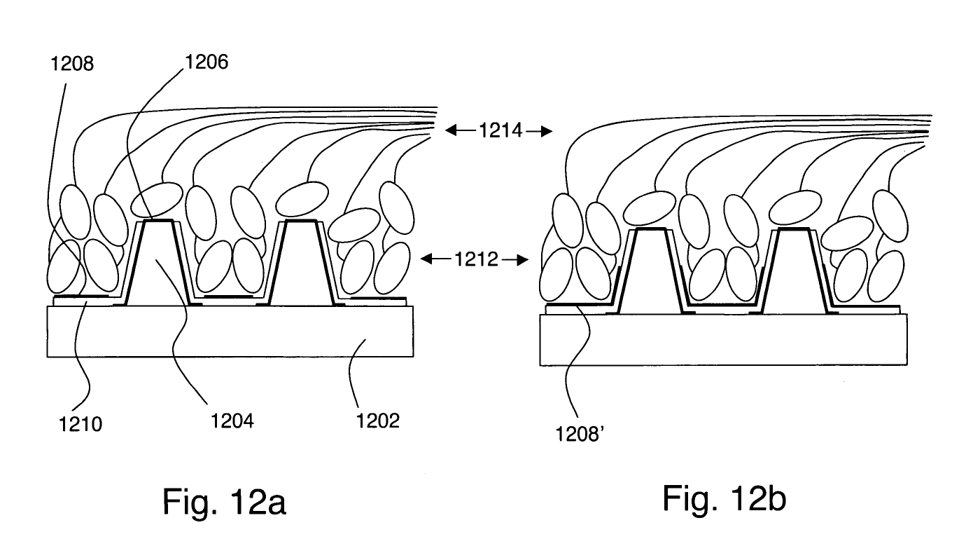

These possibilities motivate the embodiments of FIG. 12a-b. In this approach, electrodes are disposed on top of pillars to make selective contact to neural cells. More specifically, pillars 1204 are disposed on a substrate 1202. Preferably, the pillar height is between 20 μm and 200 μm, the pillar diameter is between 5 μm and 25 μm, and the lateral spacing between pillars is between 20 μm and 100 μm. Electrodes (or traces) 1206 are disposed on pillars 1204 such that the electrodes are exposed to neural cells 1212 at the tops of pillars 1204. However, the sides of pillars 1204 are electrically insulated from cells 1212 by an insulating layer 1210. Electrical insulation of the sides of the pillars provides improved excitation selectivity compared to a conventional “bed of nails” electrode array. Excitation of electrodes 1206 leads to excitation of neural cells 1212 that are in close proximity to the active electrodes. The excited neural cells then provide signals to nerve fibers 1214.

A common return electrode 1208 can be disposed on top of insulating layer 1210. In some cases, as shown on FIG. 12a, return electrodes 1208 do not extend up the sides of pillars 1204. In other cases, as shown on FIG. 12b, return electrodes 1208′ extend at least partly up the sides of pillars 1204.

Although the interface of FIGS. 12a-b can be mechanically inserted into a biological neural network, it is preferable to position the interface in close proximity to the neural network and allow or induce cellular migration to positions between the pillars. Thus the interface of FIGS. 12a-b can make selective contact to cells which migrate slowly (or do not migrate at all) without incurring the cellular injury associated with mechanical insertion of an electrode interface. Suitable methods of allowing or inducing cellular migration are described above.

One approach for fabricating the embodiment of FIGS. 12a-b is to begin with a substrate 1202 that includes circuitry (e.g., electrode bond pads, optional photosensitive circuitry, etc.) fabricated in it by conventional means. A photoresist layer is deposited and patterned to create pillars 1204. Next, a first metal layer is deposited and patterned to create electrodes 1206 connected to substrate 1202 (typically one electrode and connection is made per pixel of the electrode array). Next, an electrical insulator is deposited and patterned to create insulating layer 1210 such that the tops of the pillars are exposed and all other parts of the interface are substantially insulated. Next, a second metal layer is deposited and patterned to create a common electrode 1208 on top of insulating layer 1210. Alternatively, pillars 1204 can be fabricated from an electrically conductive material (instead of photoresist).

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art.

For example, additional perforations can be included in the membrane to assist and/or ensure flow of nutrients. The diameter of such perforations should be smaller than the diameter of the channels to avoid neural cell migration through these additional perforations (i.e., tuft formation), but large enough to ensure a flow of nutrients. Specific growth factor(s) or surface coatings can be used to ensure migration of a particular cell group, e.g. only bipolar cells, or even a specific type of bipolar cell (e.g., “on” or “off” cells). Also, the interface can have some channels or perforations for stimulation purposes while other channels or perforations can be designed for mechanical anchoring to neural tissue. Generally, interfaces according to the invention can be either optically activated or non-optically activated. Excitation with bi-phasic electrical pulses is typically preferred (but not required) in all embodiments of the invention.

The present invention is not limited to placement of the interface under the neural tissue since the interface can also be placed over or within the neural tissue. The interface can be used as a prosthetic device to connect to various kinds of neural tissue and is not limited to a retinal prosthesis or interface.

The interface has been discussed in light of electrically stimulating a select group of neural cells, however, the interface could also be used to measure signals generated in neural cells due to an external trigger/excitation, for example, signals generated in retinal cells due to light excitation.

In the discussion of FIG. 10, a preferred lithographic fabrication approach for the embodiment of FIG. 10 was discussed. Likewise, laser processing was discussed in connection with the embodiment of FIG. 6. The invention is not limited to any one fabrication method. Thus the use of lithography is not restricted to the embodiment of FIG. 10. Similarly, the use of laser processing is not restricted to the embodiment of FIG. 6.

All such variations are considered to be within the scope and spirit of the present invention as defined by the following claims and their legal equivalents.

1. An interface for selectively making electrical contact to a plurality of neural cells in a biological neural network, said interface comprising:
a) a membrane having a thickness of less than 0.5 mm and including a plurality of channels passing through said thickness of said membrane, said membrane disposed in proximity to said biological neural network, whereby said neural cells are capable of migrating into said channels;
b) a substrate in proximity to said membrane, wherein a surface of said substrate facing said membrane provides end faces for each of said channels; and
c) a plurality of first electrodes disposed on said end faces of said channels;
wherein sufficient space is present in said channels to permit migration of at least one of said neural cells into said channels.

2. The interface of claim 1, wherein said membrane thickness is in a range from about 5 microns to about 100 microns.

3. The interface of claim 1, wherein said first electrodes are in physical contact with said neural cells or spaced apart from said neural cells.

4. The interface of claim 1, wherein said biological neural network comprises a brain cortex neural network or a retinal neural network.

5. The interface of claim 1, wherein said first electrodes are connected to a plurality of photo-sensitive circuits.

6. The interface of claim 1, wherein said first electrodes are coated with a high surface area layer, whereby electrochemical erosion of said electrodes is substantially reduced.

7. The interface of claim 1, wherein said plurality of channels is arranged in a two-dimensional array.

8. The interface of claim 1, wherein each of said channels is substantially circular.

9. The interface of claim 1, wherein each of said channels has substantially uniform diameter along its length, and wherein said diameter is in a range from about 5 microns to about 50 microns.

10. The interface of claim 1, further comprising a second electrode disposed on a surface of said membrane facing said biological neural network, wherein said second electrode is common to all of said plurality of channels.

11. The interface of claim 10, wherein said second electrode is transparent.

12. The interface of claim 1, wherein said membrane comprises a first layer facing said biological neural network, and a second layer facing away from said biological neural network, and wherein each of said channels has a larger diameter in said second layer than in said first layer.

13. An interface for selectively making electrical contact to a plurality of neural cells in a biological neural network, said interface comprising:
a) a substrate;
b) a plurality of electrically conductive pillars extending from said substrate, wherein top surfaces of said pillars facing away from said substrate can make electrical contact to said neural cells, wherein side surfaces of said pillars are electrically insulated from said neural cells, and wherein said pillars are not electrically connected to each other,
wherein sufficient space is present between said pillars to permit migration of at least one of said neural cells between said pillars.

14. The interface of claim 13 further comprising a common electrode disposed partly or entirely on a surface of said substrate facing said biological neural network, wherein said common electrode is common to all of said plurality of pillars.

15. The interface of claim 14, wherein said common electrode is transparent.

16. The interface of claim 14, wherein said common electrode covers at least part of said side surfaces of said pillars and is electrically insulated from said side surfaces.

17. The interface of claim 13, wherein said side surfaces are separated from said neural cells by an insulating layer disposed on said side surfaces.

18. The interface of claim 13, wherein said substrate further comprises photo-sensitive circuits connected to said top surfaces.

19. The interface of claim 13, wherein said pillars comprise a metallic coating deposited on an insulating pillar substrate.

20. The interface of claim 13, wherein said substrate comprises silicon circuitry, wherein said pillars comprise a photoresist and electrically conductive circuit traces on top of said photoresist, and wherein said traces are electrically connected to said circuitry.
